(19) United States
(12) Patent Application Publication
(10) Pub. No.: US 2025/0149185 A1
(43) Pub. Date: May 8, 2025

(54) METHODS AND SYSTEMS FOR FEDERATED QUERYING OF GENOMIC AND PHENOTYPIC DATA

(71) Applicant: KONINKLIJKE PHILIPS N.V., EINDHOVEN (NL)

(72) Inventors: Alexander Ryan Mankovich, Cambridge, MA (US); Lei Liu, Eindhoven (NL)

(21) Appl. No.: 18/729,968

(22) PCT Filed: Jan. 9, 2023
(86) PCT No.: PCT/EP2023/050252

§ 371 Date: Jul. 18, 2024

Related U.S. Application Data
(60) Provisional application No. 63/300,655, filed on Jan. 19, 2022.

(51) Int. Cl. G16H 50/70; G06F 16/2458; G16H 10/60

(52) U.S. Cl. CPC G16H 50/70; G06F 16/2471; G16H 10/60

(57) ABSTRACT

A method (100) for federated searching, comprising: (i) providing (110) a federated search system comprising a query hub in communication with a plurality of remote servers, each of the remote servers comprising phenotype and genotype information about a plurality of individuals; (ii) receiving (120) a query from a user, the query comprising a request for: phenotype information, genotype information, and one or more attributes stored at the remote server for each individual satisfying both the request for phenotype information and the request for genotype information; (iii) distributing (130) the received query to two or more of the plurality of remote servers; (iv) receiving (140) a response to the distributed query from each of the two or more of the plurality of remote servers; (v) aggregating (150) the received responses into a single aggregated response comprising both phenotype and genotype information; and (vi) providing (160) the single aggregated response.

### FIELD OF THE INVENTION

The present disclosure is directed generally to methods and systems for federated searching of both phenotype and genotype information.

### BACKGROUND

In many legacy medical record systems, whether they utilize HL7 v2, C-CDA, FHIR, or otherwise, the presence of genomics data is still somewhat sparse. This is in part due to lack of clinical need, evidence, or cost, or to the outsourcing of testing to third-party facilities. However, the advent of precision medicine and scientific advances in the last several years has brought genomics to the forefront. Standards have generally kept up with representing results of genomic tests because these tests were designed to look for specific mutations in specific genes, a relatively small set of data to report.

However, in oncology and hereditary diseases, for example, these tests have ballooned in scale recently to inspect hundreds or thousands of genes. Soon, these tests will routinely be searching through all known (˜25,000) coding genes (whole exome sequencing), or the entire genome (whole genome sequencing). In some use cases, primarily oncology, tests may also compare sequencing results from multiple samples. Medical record systems simply were not designed for data at this scale and, much like imaging, a separate system must exist for management of genomic data. Forward-looking hospital systems have moved to bespoke systems for storing raw genomic data and test results, a small subset of which are passed back to the medical record system. However, different sites have different solutions which are not interoperable, and leveraging the genomic data for research is usually impossible on a large scale.

Thus, searching disparate sources for genotype information, much less for both phenotype and genotype information using one or only a few queries is either impractically or entirely impossible.

### SUMMARY OF THE INVENTION

Accordingly, there is a need for methods and systems that enable quick and efficient searching of a plurality of different databases for both phenotype and genotype information.

The present disclosure is directed to inventive methods and systems for federated searching. Various embodiments and implementations herein are directed to a federated search system comprising a query hub in communication with a plurality of remote servers, each of these remote servers comprising phenotype and genotype information about a plurality of individuals. The query hub receives a query from a user comprising a request for: (i) phenotype information (ii) genotype information, and (iii) one or more attributes stored at the remote server for each individual satisfying both the request for phenotype information and the request for genotype information. The query hub distributes the query to the plurality of remote servers, and receives a response to the distributed query from the remote servers. The query hub aggregates the received responses into a single aggregated response comprising both phenotype and genotype information, and provides the single aggregated response to a user via a user interface of the federated search system.

Generally in one aspect, a method for federated searching is provided. The method includes: (i) providing a federated search system comprising a query hub in communication with a plurality of remote servers, each of the remote servers comprising phenotype and genotype information about a plurality of individuals; (ii) receiving, at a query hub of the federated search system, a query from a user, the query comprising a request for: phenotype information, genotype information, and one or more attributes stored at the remote server for each individual satisfying both the request for phenotype information and the request for genotype information; (iii) distributing, by the query hub, the received query to two or more of the plurality of remote servers; (iv) receiving, by the query hub, a response to the distributed query from each of the two or more of the plurality of remote servers; (v) aggregating, by the query hub, the received responses into a single aggregated response comprising both phenotype and genotype information; (vi) providing, via a user interface of the federated search system, the single aggregated response.

According to an embodiment, the received query comprises a user authentication, and wherein the received query is only distributed to a remote server if the user authentication is or would be recognized by the remote server.

According to an embodiment, aggregating the received responses into a single aggregated response comprises both phenotype and genotype information further comprises de-duplication, filtering, and/or anonymization of one or more of the received responses.

According to an embodiment, the phenotype information about each of the plurality of individuals at the remote server is stored in FHIR format.

According to an embodiment, the query comprises a phenopacket object and a request object.

According to an embodiment, the query comprises a JSON file submitted to the query hub via an API.

According to an embodiment, each of the remote servers is configured to: (i) identify one or more of the plurality of individuals that satisfy the request for phenotype information in the distributed query; (ii) identify one or more of the plurality of individuals that satisfy the request for genotype information in the distributed query; (iii) identify one or more individuals of the plurality of individuals that satisfy both the request for phenotype information and the request for genotype information; and (iv) generate a response to the distributed query comprising the identified one or more individuals of the plurality of individuals that satisfy both the request for phenotype information and the request for genotype information, and the requested one or more attributes for each such identified one or more individuals.

According to an embodiment, the response comprises a set of FHIR objects.

According to another aspect is a system for federated searching. The system includes: a plurality of remote servers, each of the remote servers comprising phenotype and genotype information about a plurality of individuals; a user interface configured to receive a query from a user, the query comprising a request for: (i) phenotype information (ii) genotype information, and (iii) one or more attributes stored at the remote server for each individual satisfying both the request for phenotype information and the request for genotype information; and a query hub in communication with the plurality of remote servers, the query hub configured to: (i) receive the query; (ii) distribute the received query to two or more of the plurality of remote servers; (iii) receive a response to the distributed query from each of the two or more of the plurality of remote servers; (iv) aggregate the received responses into a single aggregated response comprising both phenotype and genotype information; and (v) direct the user interface to provide the single aggregated response.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a flowchart of a method for federated searching, in accordance with an embodiment.

FIG. 2 is a schematic representation of a federated search system, in accordance with an embodiment.

FIG. 3 is a schematic representation of a portion of a federated search system, in accordance with an embodiment.

FIG. 4 is a schematic representation of a portion of a federated search system, in accordance with an embodiment.

### DETAILED DESCRIPTION OF EMBODIMENTS

The present disclosure describes various embodiments of a query system and method. More generally, Applicant has recognized and appreciated that it would be beneficial to provide a system that more efficiently searches a plurality of different databases for phenotype and genotype information. According to an embodiment, a federated search system comprises a query hub in communication with a plurality of remote servers, each of these remote servers comprising phenotype and genotype information about a plurality of individuals. The query hub receives a query from a user comprising a request for: (i) phenotype information (ii) genotype information, and (iii) one or more attributes stored at the remote server for each individual satisfying both the request for phenotype information and the request for genotype information. The query hub distributes the query to the plurality of remote servers, and receives a response to the distributed query from the remote servers. The query hub aggregates the received responses into a single aggregated response comprising both phenotype and genotype information, and provides the single aggregated response to a user via a user interface of the federated search system.

According to an embodiment, the methods and systems described or otherwise envisioned herein provides a framework that can accommodate federated queries across multiple systems. While query federation can hide a data source from a user, thus making reidentification of individual samples more challenging, query federation can also be leveraged to bring the algorithms to the data, further protecting patient privacy and security and enabling compliance with various international regulations (e.g. the European Union's General Data Protection Regulation or GDPR). Each data host system in the federated paradigm leverages an architecture that facilitates interoperability between GA4GH and HL7 standards, for example, harnessing their respective strengths to support sharing of genotype and phenotype data.

According to an embodiment, the methods and systems described or otherwise envisioned herein provides a unified architecture for a genomic data system and procedures for querying its contents as well as the contents of the research-consented electronic medical record via a robust interoperable clinical exchange standard such as FHIR. This structure solves the problem of interoperability between systems, enables a single query to be distributed/federated across multiple systems, and extends the capabilities of even the most technically advanced health systems to incorporate large scale genomic and phenotypic data queries for discovery processes.

The Global Alliance for Genomics and Health (GA4GH) develops standards to enable broad access to genomic and health-related data on tens of millions of individuals. This access helps drive precision medicine but depends on tools created that leverage these interoperable standards. One key aspect of this goal is that clinical records contain genotype and phenotype information that can help support a continuous learning system, a system which provides care for individual patients and, with open and responsible consenting and credentialing, can share data to drive research to further improve that care.

One standard from the GA4GH is the Beacon network. This standard enables discovery of research-consented [and] sensitive human genetic data stored in databases. Beacon is essentially a standard RESTful API for querying genomic databases with questions such as “do you have any records with allele A at location B?” and receiving a [Yes, No] or otherwise more complex response based on available data. However, Beacon to date does not support querying for specific records, details of those records, or the ability to supplement the query with additional patient-level information such as phenotypes. In this sense, Beacon does not fully support the vision described above.

FHIR (Fast Healthcare Interoperable Resources) is an HL7 standards framework created to support real world clinical and administrative scenarios across a wide variety of contexts—mobile phone apps, cloud communications, EHR-based data sharing, server communication in large institutional healthcare providers, and much more. In essence, FHIR is a next-generation, RESTful exchange approach to clinical data interoperability, and is key to the GA4GH vision paradigm; FHIR provides interoperability between health systems and can support a wide range of standard operations, including those probing patient records for phenotypes. FHIR is also capable of representing genomic data and genomic data queries, but in practice much of the genomic data will necessarily be stored in other systems (such as a Beacon). Accordingly, FHIR can be an important component of the methods and systems described or otherwise envisioned herein.

According to an embodiment, the methods and systems described or otherwise envisioned herein provides numerous advantages over the prior art. Providing a federated query system that queries a plurality of different remote servers for both phenotype and genotype information allows for more efficient retrieval of information. This information can be important to patient care, research, and many other applications. Being able to not only find this information, but to find it quickly and efficiently, enables greater applications of the data and improves research and care outcomes, potentially saving lives.

The embodiments and implementations disclosed or otherwise envisioned herein can be utilized with any system, including but not limited to medical and research databases or systems, among other systems. For example, one application of the embodiments and implementations herein is to improve analysis systems such as, e.g., the Philips IntelliSpace® products (manufactured by Koninklijke Philips, N.V.), among many other products. However, the disclosure is not limited to these devices or systems, and thus disclosure and embodiments disclosed herein can encompass any device or system utilizing phenotype and/or genotype querying.

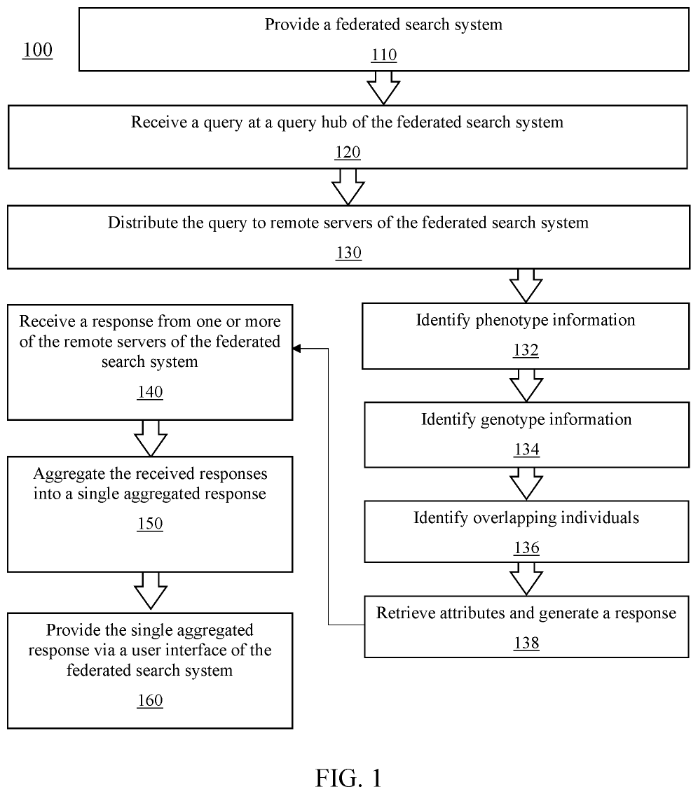

Referring to FIG. 1, in one embodiment, is a flowchart of a method 100 for federated searching using a federated search system. The methods described in connection with the figures are provided as examples only, and shall be understood not to limit the scope of the disclosure. The federated search system can be any of the systems described or otherwise envisioned herein. The federated search system can be a single system or multiple different systems.

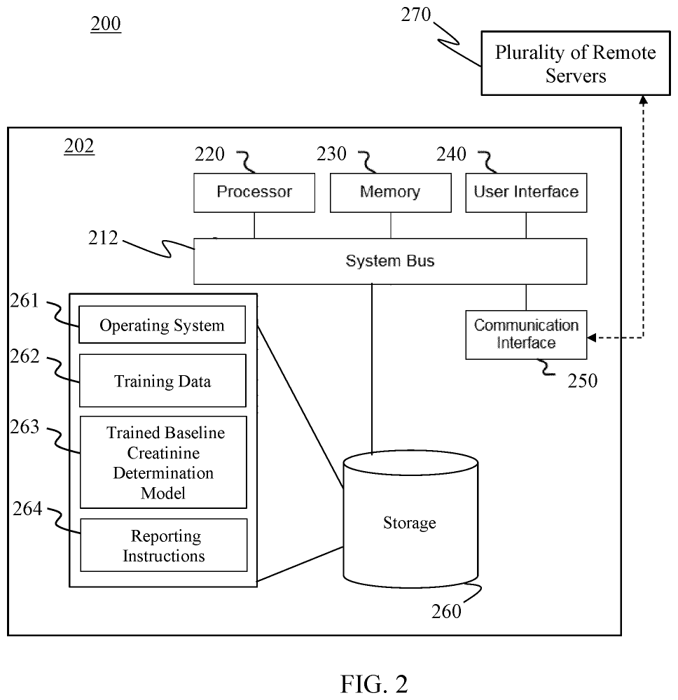

At step 110 of the method, a federated search system 200 comprising a query hub in communication with a plurality of remote servers is provided. Referring to an embodiment of a federated search system 200 as depicted in FIG. 2, for example, the system comprises one or more of a query hub 202 comprising a processor 220, memory 230, user interface 240, communications interface 250, and storage 260, interconnected via one or more system buses 212. System 200 also comprises a plurality of remote servers 270 connected via wired and/or wireless communication with the query hub 202 via communication interface 250. It will be understood that FIG. 2 constitutes, in some respects, an abstraction and that the actual organization of the components of system 200 may be different and more complex than illustrated. Additionally, federated search system 200 can be any of the systems described or otherwise envisioned herein. Other elements and components of system 200 are disclosed and/or envisioned elsewhere herein.

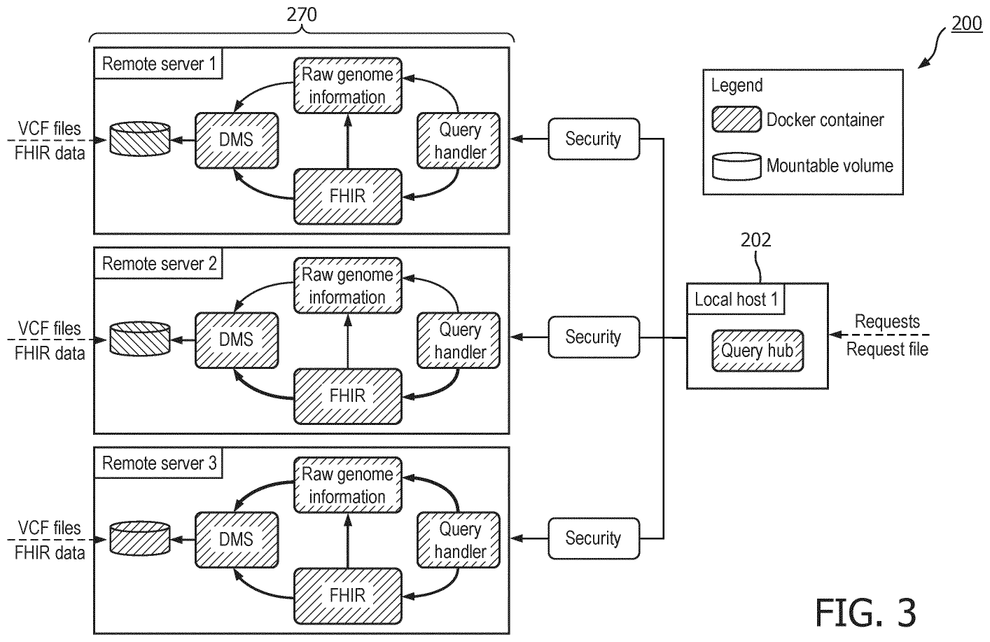

Referring to FIG. 3, in one embodiment, is a federated search system 200 comprising a query hub 202 in communication with a plurality of remote servers 270. Each remote server comprises a query handler, as well as phenotype and genotype information about a plurality of individuals. According to an embodiment, the user submits a query to the Query Hub 202 which distributes to any number of Remote Servers 270. The Remote Servers retrieve and send the results to the Query Hub which performs a variety of de-duplication, filtering, aggregation, and anonymization techniques before returning the final dataset to the user.

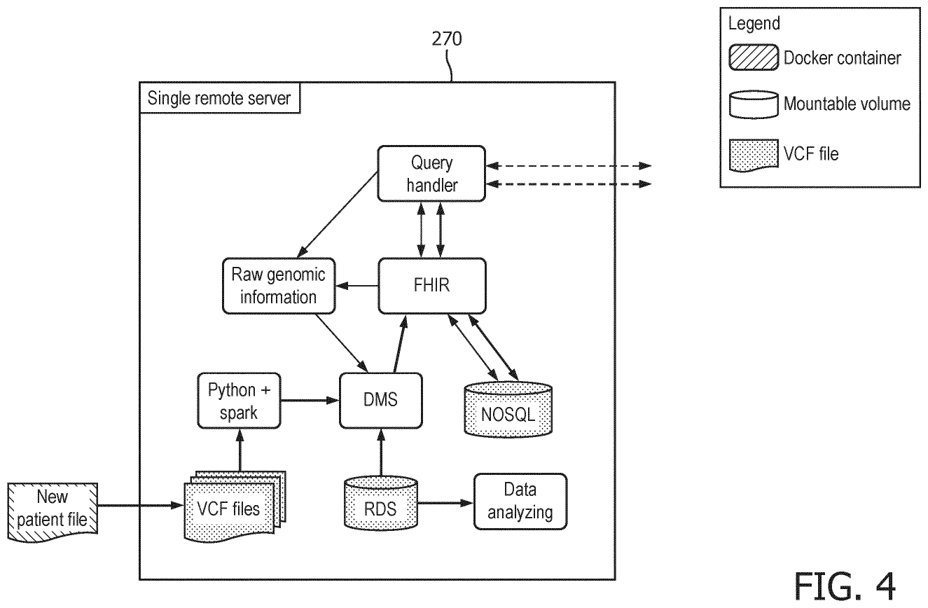

Referring to FIG. 4, in one embodiment, is one of the plurality of remote servers 270 in the federated network. According to an embodiment, the system supports incorporation of VCF files (and, with extensions, raw genomic data such as FASTQ or BAM) and FHIR data into a database management system. Each Remote Server receives simple and complex queries from the Query Hub. The server contains logic for retrieving clinical records and searching for genomic attributes in those records as well as the genomic information storage system. Clinical and genomic data are imported dynamically and automatically. The query handler is responsible for balancing queries among service containers within the Remote Server.

At step 120 of the method, the query hub receives a query from a user. The query can be provided via a user interface of the federated query system, which may be a user interface associated with the query hub, or may be a user interface that is in wired and/or wireless communication with the query hub. For example, the user may interact remotely with the query hub, such as through remote access, cloud access, or any other remote access. Accordingly, a user may be in a clinical setting, a laboratory, or in any other location and can submit a query via a local or remotely-accessed user interface. Alternatively, the user may access the system at the location of the query hub, such as at a centralized query hub location.

The query comprises, for example, a request from the user for phenotype information, genotype information, and one or more attributes for each individual satisfying both the request for phenotype information and the request for genotype information. For example, a response to the query will identify one or more people, e.g., records about one or more people, from one or more of the remote servers that satisfy both the phenotype requirement and the genotype requirement in the query. Once identified, the system will provide information for the requested one or more attributes for each of those identified people. The attribute can be any information about the person, such as a demographic, a phenotype, a genotype, diagnosis, treatment information, and/or any other information that can be associated in the remote server with an individual.

According to an embodiment, a query is constructed by a user such as a clinician or researcher. The query can consist of a Phenopacket object, coupled with a Request object, which can be either submitted to the Query Hub directly or, optionally, a user interface may be provided to create a Phenopacket and Request object in-place. According to an embodiment, a Phenopacket is a standard from GA4GH, and is an anonymous description of an individual or biosample with potential genes of interest and/or diagnoses. Phenopackets can support a variety of use cases, but according to one use a Phenopacket is a standard representation of genomic and phenotypic data for an individual or sample.

According to an embodiment, a query request contains basic information on the patient matching criteria and requested information for matching records. These queries, which may be defined in a standard JSON file generated by the user, can be submitted to the Query Hub through a unified API. This JSON may itself leverage existing standard representations for the matching criteria (clinical and genomic data), such as Phenopackets, as well as any standard representations stating the requested information. The connections between the Query Hub and Remote Servers are secured and authorized. Sources of each database in Remote Servers are VCF files (though extensions can be made to incorporate other genomic data formats such as FASTQ, SAM, BAM, or downstream formats representing gene expression and other data) and patient information in FHIR format. Every module is containerized and can be replaced or scaled up more easily.

According to an embodiment, the contents of the Phenopacket are limited to the Phenopacket specification in order to ensure interoperability, though several fields in the specification would likely go unused on the query side of this system (seeing as the general goal is to build a cohort, fields such as ‘subject’ and ‘biosamples’ are only relevant when returning results). However, to maximize flexibility for follow-up queries, and assuming the system has proper consenting and credentialing procedures, these fields remain an option. The Phenopacket would usually contain an ID (reference for the Phenopacket itself), phenotypic features, biosample, genes, variants, and diseases. The contents of each field (some of which are objects themselves) is limited to the Phenopacket specification as well.

While the Phenopacket describes the cohort the user is looking for, the user needs to specify what data they want out of that cohort. The data the user wants to retrieve is conditional and limited to the data they have access to and data that is consented to by the patient for the intended purpose of the user. The Request object itself would generally apply to clinical data the user would like to retrieve based on the members of the desired cohort. In order to interoperate with where phenotypic data is stored, either within or able to be constructed in a FHIR environment, the Request object would comply with a FHIR search object.

Below is an example of a query to a FHIR server for all FHIR Resources with a particular condition (with a tag of 12345). Implementations of this invention would typically ensure that any requested data was in compliance with user credentials and patient consents prior to delivery to the query server.
GET [base]/Condition?_tag-http://acme.org/codes|12345

According to an embodiment, therefore, the user generates a JSON file containing the Phenopacket and Request objects, and sends the request through a unified URL to the Query Hub. Authentication may be controlled by adding a token at the header of each request or through other authentication and access control mechanisms. One such other authentication mechanism could be an API request to a separate service, such as one leveraging the GA4GH Data Use & Researcher Identities.

At step 130 of the method, the query hub distributes the received query to at least some, and possibly all, of the plurality of the remote servers. There may be a preliminary check by which the query hub decides which of the plurality of remote servers to which to send the query. For example, according to an embodiment, the received query can comprise a user authentication, and the received query may only be distributed to a remote server if the user authentication is or would be recognized by the remote server. Thus, a user may require authorization to search a remote server.

According to an embodiment, each Remote Server receives GET requests (POST methods are not supported) from the Query Hub. These requests contain information on the type of patient to find (defined in the Phenopackets object) and the data to extract from those patients. The phenotype information (from the Phenopacket) is first used to identify patients in the FHIR server matching the user description. This is done by searching through a variety of Resource types (such as Condition, Observation, DiagnosticReport) in the FHIR server for matching attributes, facilitated by the FHIR GET request. In tandem, the genotype information (also from the Phenopacket) is used to query the Beacon server. The matching records are mapped to patients in the FHIR server, and an intersection is performed to obtain the patient records matching both the phenotype and genotype attributes in the Phenopacket. Next, the Request object is used together with the resulting patient records (i.e., resource IDs) to generate another FHIR GET request to obtain the requested attributes for each patient. These attributes may be quantitative (such as age) or categorical (such as disease, administered therapies, outcomes) in nature and are then returned to the Query Hub.

Accordingly, at step 132 of the method, the remote server identifies one or more of the plurality of individuals for which information is stored at the server that satisfy the request for phenotype information in the distributed query. Similarly, at step 134 of the method, the remote server identifies one or more of the plurality of individuals that satisfy the request for genotype information in the distributed query. Notably, steps 132 and 134 may be done in any order sequentially, or may be performed simultaneously.

At step 136 of the method, the remote server identifies one or more individuals of the plurality of individuals that satisfy both the request for phenotype information and the request for genotype information. For example, a remote server may identify 50 people that satisfy the phenotype requirement in the query, and may identify 100 people that satisfy the genotype requirement in the query. The remote server will then look for the overlap within the 50 people and the 100 people, and may identify a total of 10 people that satisfy both the phenotype requirement in the query and the genotype requirement in the query.

At step 138 of the method, the remote server generates a response to the distributed query, which can then be returned to the query hub. The response comprises information about the identified one or more individuals of the plurality of individuals that satisfy both the request for phenotype information and the request for genotype information. The response also comprises information about the one or more requested attributes from the query for each of the identified one or more individuals of the plurality of individuals that satisfy both the request for phenotype information and the request for genotype information.

According to an embodiment, to support an even more secure paradigm, Remote Servers may choose only to respond to requests containing untrained models. In these setups, the user provides the Phenopacket and Request objects together with a Model object (an untrained learning model). The Query Hub will distribute this package to Remote Servers configured to accept Model objects, iteratively: the package is sent to the first Remote Server, and a trained model is returned; the same package with the trained model is sent to the second Remote Server, and a further trained model is returned; and so on. Due to the nature of the federated querying system, only some learning models are suitable: incremental learning models. These models are capable of training on blocks of data, one after the other, and thus can be trained over a series of Remote Servers.

In addition to delivery of the models, the user will want to specify how to handle pre-processing of the (hidden) data (performing normalization, methods to handle missing values), and as such the Remote Server will need a machine learning framework to handle the model and other requirements.

All preprocessing functions (such as data cleaning, feature extraction, etc.) can be encapsulated into a single container which collects data from other containers inside the Remote Server. The model and data configurations can be described in the metadata of the platform or uploaded by the user to the Query Hub in JSON format. All Remote Server results are transmitted through query handler inside container and filtered by the Query Hub.

At step 140 of the method, the query hub receives a response to the distributed query from each of the plurality of remote servers. A response may comprise a fully responsive answer comprising all the requested information, or may indicate that there is no information at the server responsive to the request. A response may also comprise something between those two responses, such as a partially responsive answer with only some of the requested information. The received response may be utilized immediately, or may be stored for future processing.

At step 150 of the method, the query hub aggregates the received responses into a single aggregated response comprising both phenotype and genotype information, as well as the responsive attribute information. According to an embodiment, aggregating the received responses into a single aggregated response comprising both phenotype and genotype information further comprises de-duplication, filtering, and/or anonymization of one or more of the received responses.

According to an embodiment, results from each Remote Server are collected as a set of FHIR objects (Resources) and returned from each host to the Query Hub. The Query Hub's primary responsibility is to aggregate the total set of results from each host into a single Result object. Secondary responsibilities include any number of important post-processing events, including identification and subsequent removal or merging of duplicate records or additional anonymization techniques (such as k-anonymity). After the Result object is constructed and processed by the Query Hub, it is finally sent to the user.

At step 160 of the method, the federated search system provides the single aggregated response to the user via a user interface of the system. The response may comprise any information that is returned in response to the query, including attribute information, individual information, phenotype information, genotype information, and more. The single aggregated response can be provided via the user interface using any method for conveying or displaying information, and the user interface can be any device, interface, or mechanism for providing the conveyed or displayed information. For example, the single aggregated response to the user's query may be communicated by wired and/or wireless communication to another device. For example, the system may communicate the information to a mobile phone, computer, laptop, wearable device, and/or any other device configured to allow display and/or other communication of the information. The user interface can be any device or system that allows information to be conveyed and/or received, and may include a display, a mouse, and/or a keyboard for receiving user commands.

Referring to FIG. 2 is a schematic representation of a federated query system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.\

According to an embodiment, system 200 comprises a plurality of remote servers 270, which may be any of the remote servers described or otherwise envisioned herein. The remote servers can comprise different databases, and each database can comprise phenotype and genotype information about individuals, as well as other attributes about individuals.

According to an embodiment, system 200 comprises a query hub 202. According to an embodiment, query hub 202 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of query hub 202. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may be located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 and/or query hub 202 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 and/or query hub 202 is implemented in a cloud computing system, the various hardware components may belong to separate physical systems. For example, processor 220 may include a first processor in a first server and a second processor in a second server. Many other variations and configurations are possible.

According to an embodiment, storage 260 of query hub 202 may store one or more algorithms, modules, and/or instructions to carry out one or more functions or steps of the methods described or otherwise envisioned herein. For example, the system may comprise, among other instructions or data, distribution instructions 262, aggregation instructions 263, and/or reporting instructions 264, among many other possible instructions and/or data.

According to an embodiment, distribution instructions 262 direct the system to receive a query from a user interface and/or distribute a query to the plurality of distributed remote servers, as described or otherwise envisioned herein. There may be a preliminary check by which the query hub decides which of the plurality of remote servers to which to send the query. For example, according to an embodiment, the received query can comprise a user authentication, and the received query may only be distributed to a remote server if the user authentication is or would be recognized by the remote server. Thus, a user may require authorization to search a remote server.

According to an embodiment, aggregation instructions 263 direct the system to receive a response from the plurality of remote servers, and/or aggregate the received response into a single aggregated response comprising both phenotype and genotype information, as well as the responsive attribute information, as described or otherwise envisioned herein. According to an embodiment, aggregating the received responses into a single aggregated response comprising both phenotype and genotype information further comprises de-duplication, filtering, and/or anonymization of one or more of the received responses.

According to an embodiment, reporting instructions 264 direct the system to generate and provide a report or visualization to a user via the user interface 240 of the federated search system 200, as described or otherwise envisioned herein. The response may comprise any information that is returned in response to the query, including attribute information, individual information, phenotype information, genotype information, and more. The single aggregated response can be provided via the user interface using any method for conveying or displaying information, and the user interface can be any device, interface, or mechanism for providing the conveyed or displayed information. For example, the single aggregated response to the user's query may be communicated by wired and/or wireless communication to another device. For example, the system may communicate the information to a mobile phone, computer, laptop, wearable device, and/or any other device configured to allow display and/or other communication of the information. The user interface can be any device or system that allows information to be conveyed and/or received, and may include a display, a mouse, and/or a keyboard for receiving user commands.

### EXAMPLES

The following are non-limiting examples of implementations of the federated search systems and methods described or otherwise envisioned herein.

According to a first example, all containers can be configured and brought up in a ‘docker-compose.yml’ file. For the Remote Server, VCF and FHIR files are imported and a port is opened to accept connections from the Query Hub. The Query Hub can be deployed in another host with the appropriate IP addresses of remote servers. Permissions on shared folders should be carefully configured.

According to a second example, a researcher studying the demographics of breast cancer would like to test a hypothesis regarding breast cancer survival rates across different age groups. The researcher has valid credentials to access numerous data servers in the federated research network. The researcher has collected the phenotype (disorder=‘breast cancer’) they would like to select patients for, and packages that phenotype into a Phenopacket along with a Request object (specifying the age, ethnicity, and gender) into a single JSON. Additionally, they provide an untrained incremental learning model called SGDRegressor (a linear regression model) as well as preprocessing instructions for the data (e.g., replace missing numerical values by the mean of all values for that type, and replace missing categorical values with the most frequent value for that type, using one-hot encoding for categorical features, etc.). These inputs are submitted to the Query Hub, where these instructions are carried out incrementally. The Query Hub will skip Remote Servers that do not accept the researcher's credentials. Finally, once all Remote Servers have responded (with or without the requested model), the trained model is returned to the user for further testing.

According to a third example, a clinician has a patient with an undiagnosed disorder and would like to leverage the federated research network to find similar patients that may have a clinical diagnosis. The clinician interacts with the Query Hub via a web application where they enter the phenotypes of his patient, and the requested diagnosis attribute. They submit their query and two unique diagnoses are returned to the clinician. Upon further review of medical literature for each, the clinician has high confidence that the patient has one of the two diseases, and arranges a proper treatment regimen.

According to a fourth example, a pharmaceutical company is working to develop a drug to treat a particular type of patient. However, it was recently discovered that the drug may be ineffective and dangerous in patients with a particular genotype. To determine whether the eligible patient population is still large enough to justify high development costs, the pharmaceutical company needs to assess the frequency of these patients with the genotype. The technical division submits a query containing all of the eligibility criteria in a Phenopacket object, and the Request object containing the genotype (a particular genetic variant). The Query Hub distributes the query and returns binary results for all patients matching the eligibility criteria; upon further analysis, the pharmaceutical company discovers that an unacceptable proportion of patients has the stated genotype and decides to halt development of the drug.

The searching of a genomic dataset for genomic information, much less searching of multiple genomic datasets at one of the remote servers of the federated search system, comprises millions or billions of calculations, something the human mind is not equipped to perform, even with pen and pencil. Indeed, a genomic dataset alone comprises millions of pieces of information. For example, next-generation DNA sequencing data comprises reads that number in the 100s of millions or even billions.

Further, the methods described herein significantly improve the speed and functionality of a distributed search system. For example, by implementing the methods described herein, the federated search system is able to search disparate databases much faster and more efficiently than prior art systems. Prior art systems cannot provide this functionality, and therefore are inferior systems. Accordingly, the methods described herein significantly improve the speed and functionality of a search system.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for federated searching, comprising:
providing a federated search system comprising a query hub in communication with a plurality of remote servers, each of the remote servers comprising phenotype and genotype information about a plurality of individuals;
receiving, at a query hub of the federated search system, a query from a user, the query comprising a request for: (i) phenotype information, (ii) genotype information, and (iii) one or more attributes stored at the remote server for each individual satisfying both the request for phenotype information and the request for genotype information;
iteratively distributing, by the query hub, the received query together with an incremental learning model to two or more of the plurality of remote servers;
iteratively receiving, by the query hub, a response to the distributed query together with a trained version of the incremental learning model from each of the two or more of the plurality of remote servers; wherein said iteratively distributing and receiving comprises sending the received query together with an untrained incremental learning model to a first remote server and receiving together with the response a first trained version of the incremental learning model and sending the received query together with the first trained version of the incremental learning model to a second remote server and receiving a further response and a second trained version of the incremental learning model;
aggregating, by the query hub, the received responses into a single aggregated response comprising both phenotype and genotype information; and
providing, via a user interface of the federated search system, the single aggregated response and a latest trained version of the incremental learning model.

2. The method of claim 1, wherein the received query comprises a user authentication, and wherein the received query is only distributed to a remote server if the user authentication is or would be recognized by the remote server.

3. The method of claim 1, wherein aggregating the received responses into a single aggregated response comprises both phenotype and genotype information further comprises de-duplication, filtering, and/or anonymization of one or more of the received responses.

4. The method of claim 1, wherein the phenotype information about each of the plurality of individuals at the remote server is stored in FHIR format.

5. The method of claim 1, wherein the query comprises a phenopacket object and a request object and a model object comprising the incremental learning model.

6. The method of claim 5, wherein the query comprises a JSON file submitted to the query hub via an API.

7. The method of claim 1, wherein each of the remote servers is configured to:
identify one or more of the plurality of individuals that satisfy the request for phenotype information in the distributed query;
identify one or more of the plurality of individuals that satisfy the request for genotype information in the distributed query;
identify one or more individuals of the plurality of individuals that satisfy both the request for phenotype information and the request for genotype information; and
generate a response to the distributed query comprising the identified one or more individuals of the plurality of individuals that satisfy both the request for phenotype information and the request for genotype information, and the requested one or more attributes for each such identified one or more individuals.

8. The method of claim 7, wherein the response comprises a set of FHIR objects.

9. A system for federated searching, comprising:
a plurality of remote servers, each of the remote servers comprising phenotype and genotype information about a plurality of individuals;
a user interface configured to receive a query from a user, the query comprising a request for: (i) phenotype information, (ii) genotype information, and (iii) one or more attributes stored at the remote server for each individual satisfying both the request for phenotype information and the request for genotype information;
a query hub in communication with the plurality of remote servers, the query hub configured to: (i) receive the query; (ii) iteratively distribute the received query together with an incremental learning model to two or more of the plurality of remote servers; (iii) iteratively receive a response to the distributed query together with a trained version of the incremental learning model from each of the two or more of the plurality of remote servers; wherein said iteratively distributing and receiving comprises sending the received query together with an untrained incremental learning model to a first remote server and receiving together with the response a first trained version of the incremental learning model and sending the received query together with the first trained version of the incremental learning model to a second remote server and receiving a further response and a second trained version of the incremental learning model (iv) aggregate the received responses into a single aggregated response comprising both phenotype and genotype information; and (v) direct the user interface to provide the single aggregated response and a latest trained version of the incremental learning model.

10. The system of claim 9, wherein the received query comprises a user authentication, and wherein the received query is only distributed to a remote server if the user authentication is or would be recognized by the remote server.

11. The system of claim 9, wherein aggregating the received responses into a single aggregated response comprising both phenotype and genotype information further comprises de-duplication, filtering, and/or anonymization of one or more of the received responses.

12. The system of claim 9, wherein the phenotype information about each of the plurality of individuals at the remote server is stored in FHIR format.

13. The system of claim 9, wherein the query comprises a phenopacket object and a request object and a model object comprising the incremental learning model.

14. (canceled)

15. The system of claim 9, wherein each of the remote servers is configured to:
identify one or more of the plurality of individuals that satisfy the request for phenotype information in the distributed query;
identify one or more of the plurality of individuals that satisfy the request for genotype information in the distributed query;
identify one or more individuals of the plurality of individuals that satisfy both the request for phenotype information and the request for genotype information; and
generate a response to the distributed query comprising the identified one or more individuals of the plurality of individuals that satisfy both the request for phenotype information and the request for genotype information, and the requested one or more attributes for each such identified one or more individuals.

16. The method of claim 1, wherein the incremental learning model is a linear regression model, such as a SGDRegressor model.
